(19) United States
(12) Patent Application Publication
(10) Pub. No.: US 2005/0010133 A1
(43) Pub. Date: Jan. 13, 2005

(54) Specimen collection system

(76) Inventors: Larry Pestes, Boring, OR (US); Dale Pestes, Gresham, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 10/898,529

(22) Filed: Jul. 22, 2004

Related U.S. Application Data
(63) Continuation-in-part of application No. 10/615,712, filed on Jul. 8, 2003.

(51) Int. Cl. A61B 10/00

(52) U.S. Cl. 600/572

(57) ABSTRACT

A specimen collection system includes a swab having an elongate handle with an absorbent fabric tip releasably attached to one end. A container has an opening, located at one end that the swab can be inserted into. A stop, which covers a portion of the opening, has a channel which receives the handle when the swab is placed in the container. The channel is sized to allow the handle to be pulled through it out of the container, but not allow the tip to pass. Thus,. the tip is displaced from the swab and drops into the container when the handle is pulled out of the container.

### RELATED APPLICATIONS

This is a Continuation-In-Part of application Ser. No. 10/615,712 entitled SWAB HAVING EASILY DISPLACEABLE TIP FOR COLLECTING SALIVA SAMPLE AND INSERTING IT INTO A TEST TUBE, Filed Jul. 8, 2003.

### BACKGROUND AND SUMMARY OF THE INVENTION

Swabs having an elongate rigid handle and a soft, absorbent fabric foam or cotton tip are well known in the prior art. These swabs are commonly used to collect samples from a patient for testing. However, the sample often is deposited into a collection device, such as a test tube, which is too small to receive the entire swab. Co-pending patent application Ser. No. 10/615,712 discloses a swab comprising an elongate handle with a swab tip releasably attached to one end. A sleeve, which fits slidably on the handle, is moved along the handle to displace the tip off of the handle after the tip end of the swab is placed into a collection device. This allows the tip to be removed from the handle and placed in a collection device which is not large enough to receive the entire swab, without having to touch the tip. However, it is difficult for one person to hold both the collection device and the handle and still slide the sleeve down the handle to remove the tip.

The subject invention provides a collection system in which a swab tip that is releasably attached to an elongate handle can be removed from the handle and deposited into a collection device easily by one person. The system includes a swab having an elongate handle with a swab tip releasably attached to one end. A container has an opening which allows the swab to be inserted into it. A stop, which covers a portion of the opening, has a channel located in it which receives the handle when the tip is located in the container. The channel is sized to allow the handle to be pulled back out of the container through it but not allow the swab tip to be pulled through it. Thus, when the handle is pulled out of the container the tip is removed from the handle and deposited into the container.

The foregoing and other objectives, features, and advantages of the invention will be more readily understood upon consideration of the following detailed description of the invention, taken in conjunction with the accompanying drawings.

### BRIEF DESCRIPTION OF THE SEVERAL VIEWS OF THE DRAWINGS

FIG. 1 is a side elevation view of a swab which is a portion of the subject invention.

FIG. 2 is an exploded perspective view of a container which is another portion of the subject invention.

FIGS. 3 and 4 are foreshortened cross-sectional views showing how the system of the subject invention is used.

### DETAILED DESCRIPTION OF THE PREFERRED EMBODIMENT

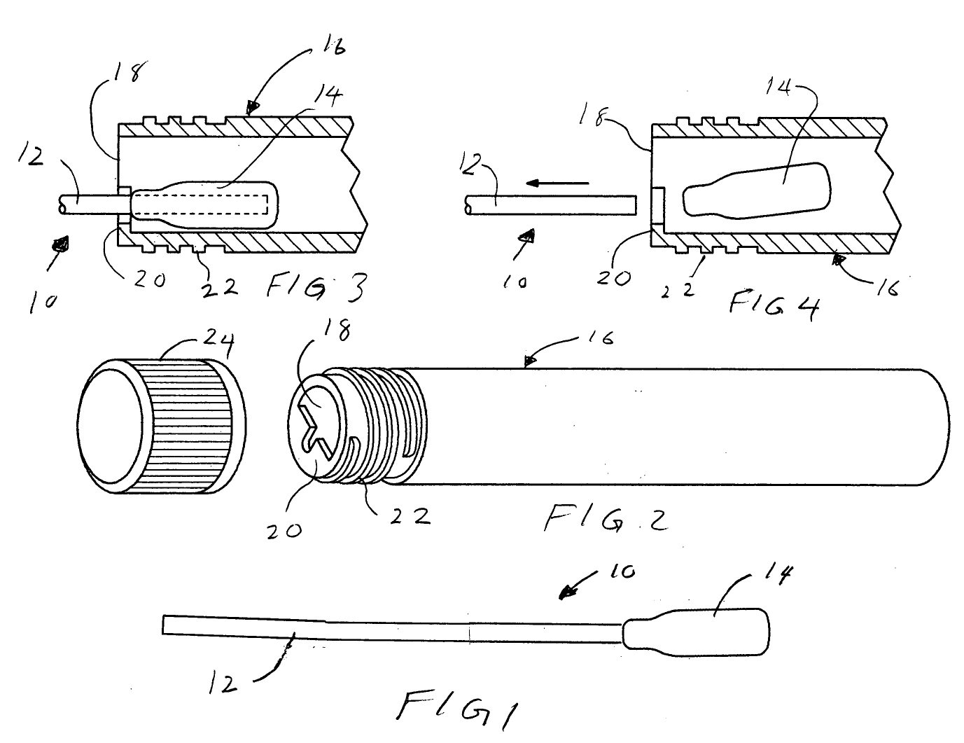

Referring now to FIG. 1 of the drawings, a swab 10 has an elongate handle 12 which generally is circular in cross-section. The swab is used to collect a sample from a patient, such as a saliva specimen, for use in a medical test. The handle is relatively rigid and preferably is made from polystyrene or a similar plastic material. In a preferred embodiment intended for collecting saliva specimens the handle is approximately 5½ inches long, is hollow and has an outside diameter of approximately 0.10 inches and an inside diameter of approximately 0.055 inches. However, the diameter of the handle and the material it is made from is not critical and the size and material will depend on the type of sample being collected.

Located on one end of the handle is an absorbent fabric tip 14. In a preferred embodiment the tip 14 is made by wrapping a thin strip of Dacron material onto the end of the handle. The Dacron material is kept under tension as it is wrapped onto the handle. After the Dacron material is wound onto the handle the tip is clamped into a heated mold to shape it and bind it together in a teardrop shape. With one exception the tip is fabricated the same way as normal fabric swab tips are. This exception is that no glue or other binder is used and the tip is maintained on the handle entirely by friction created from winding the Dacron material onto the handle while it is under tension and by whatever adhesive effect is achieved due to the application of heat and pressure. While the tip described is made by winding Dacron onto the handle, other techniques for forming fabric tips could be used, or cotton or foam tips could be used. All that is required is that the tip be easily removed from the handle but remains firmly in place on the handle during use.

A container 16, FIG. 2, has an opening 18 at one end. A portion of the opening is covered by a stop 20. The opening and stop are sized to allow the tip end of the swab to be inserted into the container, FIG. 3. In the embodiment illustrated the container is cylindrical and the end containing the opening has threads 22 formed in its outer surface. A cap 24, containing mating openings (not shown) can be placed over the open end of the container to cover the opening 18.

Located in the stop is a channel 26, which in the embodiment illustrated is U-shaped. The channel is large enough for the handle 12 to be placed in it and slid along the center line of the container, but is too small for the tip 14 to pass through it. In use, the tip end of the swab 10 is inserted into the container and the handle placed in the channel 26, FIG. 3. The handle is then pulled back out of the container until the tip strikes the stop 20. Since the tip will not pass through the channel 26 it is removed from the handle 12 as the handle is pulled further out of the container, FIG. 4. The cap 24 is then placed on the container and the container is ready to ship to a laboratory where the specimen will be analyzed. This process can easily be accomplished by a single person by holding the handle in one hand and the container in the other.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. A specimen collection system comprising:
(a) a swab having an elongate handle, and a tip which is releasably attached to said handle;
(b) a container having an opening which allows said swab to be inserted into said container;
(c) a stop which covers a portion of said opening;
(d) said stop having a channel defined therein which receives said elongate handle when said tip is located within said container; and
(e) said channel being sized such that said elongate handle can be pulled through said channel out of said container and said tip will not pass through said channel.

2. The specimen collection system of claim 1 including a cap which removably covers the end of said container containing said opening.

3. The specimen collection system of claim 1 wherein said tip is an absorbent fabric material.

4. The specimen collection system of claim 3 wherein said tip is secured to said handle only by friction created by winding a strip of fabric material onto said handle while said strip of fabric is in tension.

5. The specimen collection system of claim 1 wherein said tip is cotton.

6. The specimen collection system of claim 1 wherein said tip is foam.

7. The specimen collection system of claim 1 wherein said container is cylindrical and said channel is a U-shaped slot.

8. The specimen collection system of claim 7 wherein said container has threads located at the end thereof which contains said opening and said cap contains matching threads.
